(19) United States
(12) Patent Application Publication
(10) Pub. No.: US 2005/0037048 A1
(43) Pub. Date: Feb. 17, 2005

(54) Medical devices containing antioxidant and therapeutic agent

(76) Inventor: Young-Ho Song, Framingham, MA (US)

Correspondence Address: MAYER, FORTKORT & WILLIAMS, PC, 251 NORTH AVENUE WEST, 2ND FLOOR, WESTFIELD, NJ, 07090, US

(21) Appl. No.: 10/638,920

(22) Filed: Aug. 11, 2003

(51) Int. Cl. A61F 2/00

(52) U.S. Cl. 424/423

(57) ABSTRACT

An medical device which comprises (a) a medical device substrate and (b) a therapeutic-agent-containing region over the substrate that comprises a therapeutic agent and an antioxidant. Exemplary medical devices are implantable or insertable medical devices, such as catheters, guide wires, balloons, filters, stents, stent grafts, vascular grafts, vascular patches and shunts. Also described are methods of making devices such as those above, which methods comprise: (a) providing a solution comprising (i) solvent, (ii) the therapeutic agent, and (iii) the antioxidant; (b) providing the medical device substrate; (c) contacting the solution with the medical device substrate; and (d) removing the solvent from the solution to form the therapeutic-agent-containing region.

### FIELD OF THE INVENTION

The present invention relates to medical devices and more particularly to implantable or insertable medical devices containing oxidation-sensitive therapeutic agents.

### BACKGROUND OF THE INVENTION

Implantable or insertable medical devices are frequently used for delivery of one or more therapeutic agents. For example, an implantable or insertable medical device, such as a stent or catheter, may be provided with a coating layer that contains a therapeutic agent. Once the medical device is placed at the desired location within a patient, the therapeutic agent is released from the medical device into the patient, thereby achieving a therapeutic outcome.

Many therapeutic agents, however, are oxidation-sensitive. This characteristic can adversely impact the shelf life and efficacy of medical devices containing such therapeutic agents.

### SUMMARY OF THE INVENTION

Accordingly, there is presently a need for therapeutic-agent-containing, medical devices, in which the therapeutic agent or agents contained therein are rendered resistant to the detrimental effects of oxidation.

In this connection, the present invention is directed to novel therapeutic-agent-containing medical devices, in which an antioxidant is used to extend the life of the therapeutic agent.

According to an aspect of the present invention, a medical device, for example, an implantable or insertable medical device, is provided, which comprises a medical device substrate and a therapeutic-agent-containing region over the substrate that comprises a therapeutic agent and an antioxidant. Medical devices include medical devices adapted for implantation or insertion into the coronary vasculature, peripheral vascular system, esophagus, trachea, colon, biliary tract, urinary tract, prostate or brain, such as catheters, guide wires, balloons, filters, stents, stent grafts, vascular grafts, vascular patches and shunts.

In many embodiments, the therapeutic-agent containing region comprises a polymer in addition to the therapeutic agent and antioxidant.

In many embodiments, the therapeutic-agent-containing region is a coating layer, which is, for example, disposed over a portion of the medical device substrate or completely surrounds the medical device substrate.

Exemplary antioxidants include phenolic antioxidants such as BHT, BHA, tocopherol and probucol.

Exemplary therapeutic agent include oxidation sensitive anti-thrombotic agents, anti-proliferative agents, anti-inflammatory agents, anti-migratory agents, agents affecting extracellular matrix production and organization, anti-neoplastic agents, anti-mitotic agents, anesthetic agents, anti-coagulants, vascular cell growth promoters, vascular cell growth inhibitors, cholesterol-lowering agents, vasodilating agents, and agents that interfere with endogenous vasoactive mechanisms.

The amount of the therapeutic agent that is present in unoxidized form in the medical device after three weeks of atmospheric exposure at 25° C. is typically at least 2 times greater than the amount that is present in the absence of the antioxidant, more typically at least 3 times, 10 times or more.

Other aspects of the present invention are directed to methods of making devices such as those above, which methods comprise: (a) providing a solution comprising (i) solvent, (ii) the therapeutic agent, and (iii) the antioxidant; (b) providing the medical device substrate; (c) contacting the solution with the medical device substrate; and (d) removing the solvent from the solution to form the therapeutic-agent-containing region.

The present invention is advantageous in that therapeutic-agent-containing medical devices can be provided, which are resistant to the damaging effects of oxidation upon the therapeutic agent contained therein.

Another advantage of the present invention is that therapeutic-agent-containing medical devices can be provided, which have extended shelf life.

These and other embodiments and advantages of the present invention will become immediately apparent to those of ordinary skill in the art upon review of the Detailed Description and claims to follow.

### BRIEF DESCRIPTION OF THE DRAWINGS

FIG. 1 is a bar graph illustrating % trans-retinoic acid remaining after 21 and 40 days atmospheric exposure at 25° C., for stents having a coating containing polystyrene-polyisobutylene-polystyrene block copolymer, trans-retinoic acid, and various antioxidants.

FIG. 2 is a graph illustrating % trans-retinoic acid remaining after 21 days atmospheric exposure at 25° C., for stents having a coating of polystyrene-polyisobutylene-polystyrene block copolymer, trans-retinoic acid, and various antioxidants, as a function of antioxidant concentration.

### DETAILED DESCRIPTION OF THE INVENTION

According to an aspect of the present invention, a medical device is provided, which includes a medical device substrate and a therapeutic-agent-containing region disposed over the substrate. The therapeutic-agent-containing region includes one or more therapeutic agents and one or more antioxidants. The therapeutic-agent-containing region may be disposed over the entirety of the medical device substrate or over only a portion of the medical device. In many preferred embodiments, the therapeutic-agent-containing region is in the form of a layer (e.g., a coating) that extends over a least a portion of the medical device surface. The layer can be, for example, in the form of a carrier layer (i.e., a layer which contains at least one therapeutic agent and from which the therapeutic agent is released).

If desired, a barrier layer may be disposed between the therapeutic-agent-containing region and a site of intended release to control the rate at which the therapeutic agent is released.

Preferred medical devices for use in conjunction with the present invention include implantable or insertable medical devices such as catheters (for example, renal or vascular catheters such as balloon catheters), guide wires, balloons, filters (e.g., vena cava filters), stents (including coronary vascular stents, cerebral, urethral, ureteral, biliary, tracheal, gastrointestinal and esophageal stents), stent grafts, cerebral aneurysm filler coils (including Guglilmi detachable coils and metal coils), vascular grafts, myocardial plugs, patches, pacemakers and pacemaker leads, heart valves, biopsy devices, or any coated substrate (which can comprise, for example, glass, metal, polymer, ceramic and combinations thereof) that is implanted or inserted into the body.

The medical devices of the present invention include drug delivery medical devices that are used for either systemic treatment or for the localized treatment of any mammalian tissue or organ. Non-limiting examples are tumors; organs including the heart, coronary and peripheral vascular system (referred to overall as “the vasculature”), lungs, trachea, esophagus, brain, liver, kidney, bladder, urethra and ureters, eye, intestines, stomach, pancreas, ovary, and prostate; skeletal muscle; smooth muscle; breast; cartilage; and bone.

A particularly preferred medical device for use in connection with the present invention is a vascular medical device such as a vascular stent, which delivers therapeutic agent into the vasculature for the treatment of restenosis. As used herein, “treatment” refers to the prevention of a disease or condition, the reduction or elimination of symptoms associated with a disease or condition, or the substantial or complete elimination a disease or condition. Preferred subjects are mammalian subjects and more preferably human subjects.

As noted above, one or more antioxidants are provided in the medical devices of the present invention, along with one or more therapeutic agents. The antioxidant(s) can be, for example, a primary antioxidant (e.g., an antioxidant that terminates a free radical by donating electrons or hydrogen to the free radical) or a synergistic antioxidant (e.g., an oxygen scavenger).

Antioxidants include phenolic antioxidants (i.e., antioxidants containing a six sided aromatic ring, which as defined herein can be part of a multi-cyclic ring system, having a pendent alcohol group), including hindered phenols and polyphenolic antioxidants, such as butylated hydroxyanisole (BHA), butylated hydroxytoluene (BHT), and probucol; hydroquinones such as methyl hydroquinone, tertiary-butyl hydroquinone (TBHQ) and 1-O-hexyl-2,3,5-trimethyl hydroquinone (HTHQ); nordihydroguaiaretic acid (NDGA); alkoxyphenols such as 4-tert-butoxyphenol, 4-ethoxyphenol, 3-methoxyphenol and 2-tert-butyl-4-methoxyphenol; 2,2-methylene-bis-(4-methyl-6-tert-butylphenol); tocopherols such as alpha-tocopherol (vitamin E), beta-tocopherol, gamma-tocopherol and delta-tocopherol; phenolic acids and their esters including para-coumaric acid, caffeic acid, chlorogenic acid, ferulic acid, protocatechuic acid, cinnamic acid, gallic acid, alkyl gallates (e.g., propyl, octyl, dodecyl), and para-hydroxybenzoic acid.

Other antioxidants include flavonoids, which are generally phenolic compounds, such as catechins, leucoanthocyanidins, flavanones, flavanins, flavones, anthocyanins, flavonols, flavones, isoflavones, proanthocyanidins, flavonoid, pyrocatechol derivatives, and so forth. Specific examples are catechin, quercetin and rutin.

Further antioxidants include glutathione and ascorbic acid (vitamin C), as well as its salts (e.g., sodium and calcium ascorbate) and its esters (e.g., ascorbyl palmitate and ascorbyl stearate).

The antioxidants used in connection with the present invention are commonly antioxidants approved by the United States Food and Drug Administration (USFDA) for use in food and/or drugs.

In general, the type and the amount of the antioxidant are chosen so as to provide a medical device having enhanced stability. As defined herein, a medical device exhibits “enhanced stability” when the amount of therapeutic agent that is present in the medical device in the desired form (e.g., unoxidized form) after three weeks of atmospheric exposure at 25° C. is at least 1.25 times (more advantageously 1.5 times, 2 times, 4 times, 6 times, 8 times, 10 times, 25 times, 50 times, 100 times, 250 times, 500 times, 1000 times, or even more) greater than the amount of therapeutic agent that is present in unoxidized form in the device in the absence of the antioxidant.

Typically, the therapeutic agent in the medical device is an oxidation-sensitive therapeutic agent. A therapeutic agent within a medical device of the present invention is considered to be “oxidation sensitive” when, in the absence of antioxidant, at least 25% of the therapeutic agent in the medical device converts to another form (typically one or more oxidized forms) after three weeks of atmospheric exposure at 25° C.

Examples of oxidation-sensitive therapeutic agents include trans-retinoic acid and rapamycin. Therapeutic agents for use in connection with the present invention also include, for instance, oxidation-sensitive therapeutic agents from therapeutic agents listed below. In some embodiments, the oxidation-sensitive therapeutic agent will itself be an antioxidant, with trans-retinoic acid being one example of the same.

“Therapeutic agents”, “pharmaceutically active agents”, “pharmaceutically active materials”, “drugs” and other related terms may be used interchangeably herein and include genetic therapeutic agents, non-genetic therapeutic agents and cells. Therapeutic agents may be used singly or in combination.

Non-genetic therapeutic agents include: (a) anti-thrombotic agents such as heparin, heparin derivatives, urokinase, and PPack (dextrophenylalanine proline arginine chloromethylketone); (b) anti-inflammatory agents such as dexamethasone, prednisolone, corticosterone, budesonide, estrogen, sulfasalazine and mesalamine; (c) antineoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promotors; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; and (o) agents that interfere with endogenous vasoactive mechanisms.

Genetic therapeutic agents include anti-sense DNA and RNA as well as DNA coding for: (a) anti-sense RNA, (b) tRNA or rRNA to replace defective or deficient endogenous molecules, (c) angiogenic factors including growth factors such as acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor and insulin-like growth factor, (d) cell cycle inhibitors including CD inhibitors, and (e) thymidine kinase (“TK”) and other agents useful for interfering with cell proliferation. Also of interest is DNA encoding for the family of bone morphogenic proteins (“BMP's”), including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them.

Vectors for delivery of genetic therapeutic agents include (a) plasmids, (b) viral vectors such as adenovirus, adenoassociated virus and lentivirus, and (c) non-viral vectors such as lipids, liposomes and cationic lipids.

Cells include cells of human origin (autologous or allogeneic), including stem cells, or from an animal source (xenogeneic), which can be genetically engineered, if desired, to deliver proteins of interest.

Numerous therapeutic agents, not necessarily exclusive of those listed above, have been identified as candidates for vascular treatment regimens, for example, as agents targeting restenosis. Such agents include one or more of the following: (a) Ca-channel blockers including benzothiazapines such as diltiazem and clentiazem, dihydropyridines such as nifedipine, amlodipine and nicardapine, and phenylalkylamines such as verapamil, (b) serotonin pathway modulators including: 5-HT antagonists such as ketanserin and naftidrofuryl, as well as 5-HT uptake inhibitors such as fluoxetine, (c) cyclic nucleotide pathway agents including phosphodiesterase inhibitors such as cilostazole and dipyridamole, adenylate/Guanylate cyclase stimulants such as forskolin, as well as adenosine analogs, (d) catecholamine modulators including α-antagonists such as prazosin and bunazosine, β-antagonists such as propranolol and α/β-antagonists such as labetalol and carvedilol, (e) endothelin receptor antagonists, (f) nitric oxide donors/releasing molecules including organic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine, (g) ACE inhibitors such as cilazapril, fosinopril and enalapril, (h) ATII-receptor antagonists such as saralasin and losartin, (i) platelet adhesion inhibitors such as albumin and polyethylene oxide, 0) platelet aggregation inhibitors including aspirin and thienopyridine (ticlopidine, clopidogrel) and GP IIb/IIIa inhibitors such as abciximab, epitifibatide and tirofiban, (k) coagulation pathway modulators including heparinoids such as heparin, low molecular weight heparin, dextran sulfate and β-cyclodextrin tetradecasulfate, thrombin inhibitors such as hirudin, hirulog, PPACK(D-phe-L-propyl-L-arg-chloromethylketone) and argatroban, FXa inhibitors such as antistatin and TAP (tick anticoagulant peptide), Vitamin K inhibitors such as warfarin, as well as activated protein C, (l) cyclooxygenase pathway inhibitors such as aspirin, ibuprofen, flurbiprofen, indomethacin and sulfinpyrazone, (m) natural and synthetic corticosteroids such as dexamethasone, prednisolone, methprednisolone and hydrocortisone, (n) lipoxygenase pathway inhibitors such as nordihydroguairetic acid and caffeic acid, (o) leukotriene receptor antagonists, (p) antagonists of E- and P-selectins, (q) inhibitors of VCAM-1 and ICAM-1 interactions, (r) prostaglandins and analogs thereof including prostaglandins such as PGE1 and PGI2 and prostacyclin analogs such as ciprostene, epoprostenol, carbacyclin, iloprost and beraprost, (s) macrophage activation preventers including bisphosphonates, (t) HMG-CoA reductase inhibitors such as lovastatin, pravastatin, fluvastatin, simvastatin and cerivastatin, (u) fish oils and omega-3-fatty acids, (v) free-radical scavengers/antioxidants such as probucol, vitamins C and E, ebselen, trans-retinoic acid and SOD mimics, (w) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators such as sulotroban, vapiprost, dazoxiben and ridogrel, as well as protein tyrosine kinase inhibitors such as tyrphostin, genistein and quinoxaline derivatives, (x) MMP pathway inhibitors such as marimastat, ilomastat and metastat, (y) cell motility inhibitors such as cytochalasin B, (z) antiproliferative/antineoplastic agents including antimetabolites such as purine analogs (e.g., 6-mercaptopurine or cladribine, which is a chlorinated purine nucleoside analog), pyrimidine analogs (e.g., cytarabine and 5-fluorouracil) and methotrexate, nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, colchicine, paclitaxel and epothilone), caspase activators, proteasome inhibitors, angiogenesis inhibitors (e.g., endostatin, angiostatin and squalamine), rapamycin, cerivastatin, flavopiridol and suramin, (aa) matrix deposition/organization pathway inhibitors such as halofuginone or other quinazolinone derivatives and tranilast, (bb) endothelialization facilitators such as VEGF and RGD peptide, and (cc) blood rheology modulators such as pentoxifylline.

Numerous additional therapeutic agents are also disclosed in U.S. Pat. No. 5,733,925 assigned to NeoRx Corporation, the entire disclosure of which is incorporated by reference.

A wide range of therapeutic agent loadings can be used in connection with the medical devices of the present invention, with the amount of loading being readily determined by those of ordinary skill in the art and ultimately depending, for example, upon the condition to be treated, the nature of the therapeutic agent itself, the means by which the therapeutic agent is administered to the intended subject, and so forth.

In some embodiments of the present invention, the therapeutic-agent-containing region contains one or more polymers, in addition to therapeutic agent(s) and antioxidant(s). A variety of polymers can be used for this purpose. For example, the polymer may be a homopolymer or a copolymer (including alternating, random and block copolymers), cyclic, linear or branched (e.g., polymers have star, comb or dendritic architecture), natural or synthetic, thermoplastic or thermosetting. Polymers include the following: polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydoxyalkyl celluloses; polyoxymethylene polymers and copolymers; polyimide polymers and copolymers such as polyether block imides, polyamidimides, polyesterimides, and polyetherimides; polysulfone polymers and copolymers including polyarylsulfones and polyethersulfones; polyamide polymers and copolymers including nylon 6,6, polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinylacetate copolymers (EVA), polyvinylidene chlorides, polyvinyl ethers such as polyvinyl methyl ethers, polystyrenes, styrene-maleic anhydride copolymers, styrene-butadiene copolymers, styrene-ethylene-butylene copolymers (e.g., a polystyrene-polyethylene/butylene-polystyrene (SEBS) copolymer, available as Kraton® G series polymers), acrylonitrile-styrene copolymers, acrylonitrile-butadiene-styrene copolymers, styrene-butadiene copolymers and styrene-isobutylene copolymers (e.g., polyisobutylene-polystyrene block copolymers such as SIBS), polyvinyl ketones, polyvinylcarbazoles, and polyvinyl esters such as polyvinyl acetates; polybenzimidazoles; ionomers; polyalkyl oxide polymers and copolymers including polyethylene oxides (PEO); glycosaminoglycans; polyesters including polyethylene terephthalates and aliphatic polyesters such as polymers and copolymers of lactide (which includes lactic acid as well as d-,l- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of polylactic acid and polycaprolactone is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone polymers and copolymers; polyurethanes; p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), including fibrin, fibrinogen, collagen, elastin, chitosan, gelatin, starch, glycosaminoglycans such as hyaluronic acid; as well as blends and copolymers of the above.

The therapeutic-agent containing regions of the present invention can be formed in a variety of ways. Solvent-based techniques, in which therapeutic agent, antioxidant and/or polymer are dissolved in a solvent and the resulting mixture is subsequently used to form a layer on a medical device substrate, represent one group of techniques that can be used to form the therapeutic-agent containing regions.

Where solvent-based techniques are used, the solvent system that is selected will contain one or more solvent species. The solvent system typically is a good solvent for the therapeutic agent, antioxidant and/or polymer. The particular solvent species that make up the solvent system may also be selected based on other characteristics including drying rate and surface tension.

Preferred solvent-based techniques include, but are not limited to, solvent casting techniques, spin coating techniques, web coating techniques, solvent spraying techniques, dipping techniques, techniques involving coating via mechanical suspension including air suspension, ink jet techniques, electrostatic techniques, and combinations of these processes.

Where appropriate, the above application techniques can be repeated or combined with other application techniques to build up a layer to a desired thickness. The thickness of the layer can be varied in other ways as well. For example, in one preferred process, solvent spraying, coating thickness can be increased by modification of coating process parameters, including increasing spray flow rate, slowing the movement between the substrate to be coated and the spray nozzle, providing repeated passes and so forth.

In some embodiments, antioxidant and/or therapeutic agent is/are dissolved or dispersed in the solvent, along with a polymer. In other embodiments, antioxidant and/or therapeutic agent is/are dissolved within a solvent, and the resulting solution is contacted with a previously formed polymeric layer, whereupon the antioxidant and/or therapeutic agent is/are imbibed by the polymer.

Where a therapeutic-agent containing region is formed using a solvent-based technique, it is preferably dried after formation to remove the solvent species. The therapeutic-agent containing region frequently further conforms to any underlying substrate during the drying process.

It may be beneficial to maintain the therapeutic-agent containing region in a non-oxidizing environment during the course of its formation, for example, in an inert atmosphere of nitrogen and/or noble gases (e.g. helium, neon, argon, krypton etc.), to prevent oxygen from detrimentally interacting with the therapeutic agent.

It may also be beneficial to maintain the medical device in a non-oxidizing environment subsequent to its formation. For example, the medical device can be placed into packaging that has been evacuated or into which an inert gas has been introduced.

Beneficial packing materials include barrier materials which have sufficient barrier properties to maintain a vacuum or an inert gas atmosphere. Such barrier materials are known in the art.

The examples provided below are offered for illustrative purposes only, and are not intended to limit the scope of the present invention in any way. These examples relate to trans-retinoic acid (TRA) containing medical devices. Because TRA regulates proliferation, migration, differentiation, and extracellular matrix turnover of human arterial smooth muscle cells, TRA is an ideal candidate for use in medical devices, such as stents, which contain therapeutic agent for the treatment or inhibition of restenosis or atherosclerosis. Unfortunately, TRA is sensitive to light, oxygen and elevated temperatures. This has serious consequences, for example, for the shelf life of medical devices having coatings that contain TRA and for various processes that are used to form such devices. For instance, solvent-spraying techniques can be used to form TRA-containing coatings on medical devices. However, the therapeutic agent is commonly exposed to oxygen during solvent spraying. Similarly, therapeutic-agent-containing coatings on medical devices are frequently exposed to oxygen during storage. By including antioxidant in the coating formulation, however, the shelf life and stability of the medical device is increased relative to the same medical device in the absence of the antioxidant.

### EXAMPLE

Solutions (100 g total weight) are provided that contain (1) 99 g of tetrahydrofuran solvent; (2) 0.6-0.7 g of SIBS copolymer (i.e., polystyrene-polyisobutylene block copolymer; see, e.g., U.S. Pat. Appln. No. 20020107330, which is hereby incorporated by reference in its entirety); (3) 0.3 g TRA (trans-retinoic acid), and (4) one of the following (a) 0 g antioxidant, (b) 0.005 g BHT, (c) 0.01 g BHT, (d) 0.05 g BHT, (e) 0.1 g BHT, (f) 0.005 g tocopherol, (g) 0.01 g tocopherol and (h) 0.05 g tocopherol, (i) 0.1 g tocopherol, (j) 0.005 g probucol, (k) 0.01 g probucol, (l) 0.05 g probucol, (m) 0.1 g probucol, (n) 0.1 g BHA, (o) 0.1 g ascorbic acid, (p) 0.1 g glutathione, and (q) 0.1 g allopurinol. All solutions are prepared by combining the above ingredients together and mixing thoroughly.

The above solutions are placed in a syringe pump and fed to a spray nozzle. A stent is mounted onto a holding device parallel to the nozzle and rotated to ensure uniform coverage. (Depending on the spray equipment used, either the stent or spray nozzle can be moved while spraying, such that the nozzle moves along the component while spraying for one or more passes.) After a coating is formed, the stent is dried, for example, by placing it in a preheated oven. 50-60 coated stents are formed.

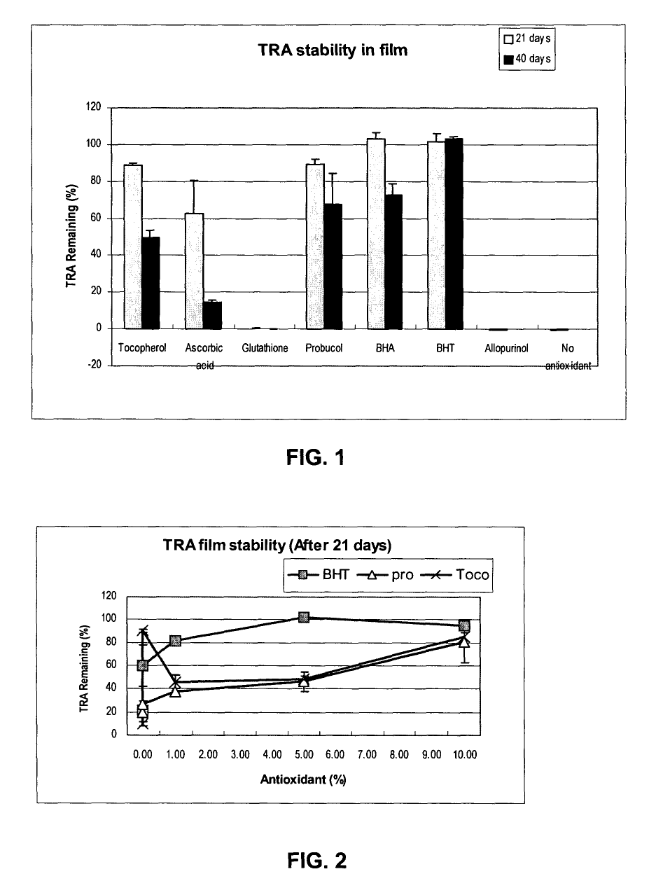

The amount of TRA present in stent formed using 0 g antioxidant and using 0.1 g of each of the seven antioxidants is then measured after 21 and 40 days and the result is presented in FIG. 1.

The amount of TRA present after 3 weeks atmospheric exposure at room temperature is also measured for stents formed using BHT, probucol and tocopherol, each in amounts of 0.0 g, 0.005 g, 0.01 g, 0.05 g, and 0.1 g. The results are presented in FIG. 2.

Although various embodiments are specifically illustrated and described herein, it will be appreciated that modifications and variations of the present invention are covered by the above teachings and are within the purview of the appended claims without departing from the spirit and intended scope of the invention.

1. An medical device comprising a medical device substrate and a therapeutic-agent-containing region over said substrate, wherein said therapeutic-agent-containing region comprises a therapeutic agent and an antioxidant.

2. The medical device of claim 1, wherein said antioxidant is a primary antioxidant.

3. The medical device of claim 1, wherein said antioxidant is a phenolic antioxidant.

4. The medical device of claim 1, wherein said antioxidant is a hindered phenolic antioxidant.

5. The medical device of claim 1, wherein said antioxidant is a hydroquinone antioxidant.

6. The medical device of claim 1, wherein said antioxidant is selected from butylated hydroxyanisole (BHA), butylated hydroxytoluene (BHT), tertiary-butyl hydroquinone (TBHQ), and 1-O-hexyl-2,3,5-trimethyl hydroquinone (HTHQ).

7. The medical device of claim 1, wherein said antioxidant is selected from tocopherol and probucol.

8. The medical device of claim 1, wherein said therapeutic-agent containing region further comprises a polymer.

9. The medical device of claim 8, wherein said polymer is a block copolymer comprising one or more polystyrene chains and one or more polyisobutylene chains.

10. The medical device of claim 8, wherein said polymer is a polystyrene-polyisobutylene-polystyrene triblock copolymer.

11. The medical device of claim 8, wherein said polymer is a biostable polymer.

12. The medical device of claim 1, wherein said therapeutic agent is selected from an anti-thrombotic agent, an anti-proliferative agent, an anti-inflammatory agent, an anti-migratory agent, an agent affecting extracellular matrix production and organization, an antineoplastic agent, an anti-mitotic agent, an anesthetic agent, an anti-coagulant, a vascular cell growth promoter, a vascular cell growth inhibitor, a cholesterol-lowering agent, a vasodilating agent, and an agent that interferes with endogenous vasoactive mechanisms.

13. The medical device of claim 1, wherein said therapeutic agent is trans-retinoic acid.

14. The medical device of claim 1, wherein said therapeutic agent is rapamycin.

15. The medical device of claim 1, further comprising a barrier region over said therapeutic-agent-containing region.

16. The medical device of claim 1, wherein said therapeutic-agent-containing region is a coating layer.

17. The medical device of claim 16, wherein said coating layer is disposed over a fraction of the medical device substrate.

18. The medical device of claim 16, wherein said coating layer completely surrounds the medical device substrate.

19. The medical device of claim 1, wherein said medical device is an implantable or insertable medical device is selected from a catheter, a guide wire, a balloon, a filter, a stent, a stent graft, a vascular graft, a vascular patch and a shunt.

20. The medical device of claim 1, wherein said medical device is a vascular stent.

21. The medical device of claim 1, wherein said medical device is an implantable or insertable medical device adapted for implantation or insertion into the coronary vasculature, peripheral vascular system, esophagus, trachea, colon, biliary tract, urinary tract, prostate or brain.

22. The medical device of claim 1, wherein said medical device is adapted for implantation or insertion into the coronary vasculature.

23. The medical device of claim 1, wherein the amount of the therapeutic agent that is present in unoxidized form in the medical device after three weeks of atmospheric exposure at 25° C. is at least 1.25 times greater than the amount of the therapeutic agent that is present in unoxidized form in the medical device after three weeks of atmospheric exposure at 25° C. in the absence of said antioxidant.

24. The medical device of claim 1, wherein the amount of the therapeutic agent that is present in unoxidized form in the medical device after three weeks of atmospheric exposure at 25° C. is at least 3 times greater than the amount of the therapeutic agent that is present in unoxidized form in the medical device after three weeks of atmospheric exposure at 25° C. in the absence of said antioxidant.

25. The medical device of claim 1, wherein the amount of the therapeutic agent that is present in unoxidized form in the medical device after three weeks of atmospheric exposure at 25° C. is at least 10 times greater than the amount of the therapeutic agent that is present in unoxidized form in the medical device after three weeks of atmospheric exposure at 25° C. in the absence of said antioxidant.
